(19) United States
(12) Patent Application Publication
(10) Pub. No.: US 2016/0015307 A1
(43) Pub. Date: Jan. 21, 2016

(54) CAPTURING AND MATCHING EMOTIONAL PROFILES OF USERS USING NEUROSCIENCE-BASED AUDIENCE RESPONSE MEASUREMENT TECHNIQUES

(71) Applicant: Ravikanth V. Kothuri, Frisco, TX (US)

(72) Inventor: Ravikanth V. Kothuri, Frisco, TX (US)

(21) Appl. No.: 14/802,511

(22) Filed: Jul. 17, 2015

Related U.S. Application Data
(60) Provisional application No. 62/025,764, filed on Jul. 17, 2014.

(51) Int. Cl. A61B 5/16; G06F 17/30

(52) U.S. Cl. CPC A61B 5/167; G06F 17/3053

(57) ABSTRACT

Disclosed is a system and method for determining the compatibility level of users by creating an emotional DNA profile for the user and matching the emotional DNA profile with profiles of other users. Based on the matching performed, appropriate content or product is displayed to the user or the level of compatibility aspect between individuals is determined. The emotional DNA profile is created by receiving inputs from various sensors that can measure user's physiological responses to content as various signals such as, facial expression, audio tone, biometrics, eyetracking and the like for various time slices and/or optionally sub-segments of standard probe content. Based on the emotional DNA profile created for the user, the overall personality is determined by optionally augmenting additional explicitly mentioned personality information of the user. Further, the emotional DNA profile that is created is matched with other users profile to determine the level of compatibility aspect between individuals.

### CROSS REFRENCE TO RELATED APPLICATION

This application claims priority to U.S. provisional application Ser. No. 62/025,764 filed Jul. 17, 2014, and entitled “CAPTURING AND MATCHING EMOTION PROFILES OF USERS USING NEUROSCIENCE-BASED AUDIENCE RESPONSE MEASUREMENT TECHNIQUES”, owned by the assignee of the present application and herein incorporated by reference in its entirety.

### FIELD OF THE INVENTION

The present invention generally relates to capturing and matching emotion profiles of users. More specifically, the present invention deals with defining and implementing a system and method for (1) measuring user responses to a pre-defined set of stimuli using neuroscience and audience-response techniques and (2) characterizing, generalizing, converting and storing such responses as user's emotional profiles for subsequent use in a variety of applications that can customize content and experience based on such pre-computed emotional profile determined for the user or to match with other appropriate users.

### BACKGROUND OF THE INVENTION

The present invention relates to capturing overall personality of a user, using both explicitly user-specified information as well as implicitly-measured neuro-signal-responses of a user to standard content, and more particularly to match user's personalities by generating emotional profile for a user, augmented with explicitly gathered information, to create a complete set that characterize the user's overall personality.

The Big Five personality traits, based on Five-Factor Model in psychology, represent five broad dimensions that are used to consistently characterize human personality. The Big Five factors are openness, conscientiousness, extraversion, agreeableness, and neuroticism. A number of researchers from 1960s to the 1980s had worked on identifying and generalizing the various traits that are common across people and arrived at almost identical (or highly correlating sets across research groups) sets and roughly agrees on the above Big-Five. Given that the Big Five traits are broad and comprehensive, they are not nearly as powerful in predicting and explaining actual behavior as are the more numerous lower-level traits. Besides, a number of researchers such as Costa and Mcrae have come up with various facets that can be deemed to constitute the Big Five traits.

In one embodiment of the invention, an association, or a relationship matching system can ‘match’ users based on the participants' big-Five traits explicitly expressed. As per the proposed invention (compared to existing methods), matching does not always have to be registering similar scores/levels on the Big-Five or even other explicitly gathered sub-dimensions. Instead, it also means complementing in nature and the level of compatibility specified by choice. For example, a user rated high on extroversion can choose to go with another user who complements him/her (that is, exactly may not be at the same level) on that dimension and hence may choose one that scores low on extroversion. Henceforth, matching refers to the type of combination by choice on the dimension (either proximity on that dimension, complete inverse on that dimension, or some degree of acceptability on that dimension specified by the users). Incorporating the Big-Five traits as additional dimensions in matching itself is one simple addition/improvement to the relationship finding.

In addition to the Big-Five traits, a number of relationship sites capture some form of personality traits using the following dimensions: eHarmony has the concept of matching on 29 dimensions which could be summarized as follows.

### Core Traits:

Emotional Temperament which is not directly related to the Big-Five but captures the self-concept, emotional energy, emotional status, and passion.
Social Status which includes dimension such as character, kindness, dominance, sociability, autonomy, and adaptability.
Cognitive dimensions such as intellect, curiosity, humor, and artistic passion.
Physicality dimensions such as energy: physical, passion: sexual, vitality and security, industry, and appearance.

### Vital Attributes:

Relationship skills such as communication style, emotion management, conflict resolution.
Value and Belief dimensions such as spirituality, family goals, traditionalism, ambition, and altruism.
Key Experience dimensions such as family background, family status, and education.

In the current scenario, all these dimensions are explicitly stated by a user and is a drawback in these type of systems for the following reasons: (1) the users may not be truly aware of the significance of these dimensions, (2) the users may not be able to measure themselves and express them correctly on the various scales for each of these dimensions, (3) the users may not be truly expressing their profile for fear of being labeled (for example, as either an introvert or so), and (4) there may be other unknown dimensions of a personality that cannot be explicitly expressed. As a consequence, the systems end up with incorrect profiles of users to start with and the existing systems fail to get the close/exact match in many cases.

### SUMMARY OF THE INVENTION

The present invention is related to a system and method for matching a user's personality and determining compatibility with the matched personality, wherein the method determines the user's personality by creating the emotional profile for the user. Further, the method determines the emotional response of the user by capturing the inputs from a variety of sensors. From such implicit probing of the inner conscience (without explicit user intervention) using neuroscience techniques, the method generates a unique emotional DNA profile for the user based on a combination of responses determined for the system-specified content stimuli (each eliciting a number of emotions). Further, the method converts the responses to an emotion-profile using proprietary algorithms either on the mobile device or by transferring the responses to a cloud-based server and transfers the responses and the profiles to/from the server to create emotion classes. Further, the method utilizes the emotion profiles to match across various users by appropriately combining the weights on the dimensions (either set by the system, or as an advanced option to be specified by the users) and prioritizing the users based on the weighted dimensions. The specific weighting of the dimensions may depend on the application. Further, based on the close match found for the emotion-profiles, the method presents appropriate marketing content/products for the user by augmenting additional information on what interests each emotion class of the users and in the relevant applications. Further, the method allows the emotional profile of the user to be visible to other users based on the user's preference.

Other objects and advantages of the embodiments herein will become readily apparent from the following detailed description taken in conjunction with the accompanying drawings. The proposed invention integrates a number of emotional and cognitive responses (including but not limited to implicit responses such as facial coding, biometrics, eye tracking, voice emotion as well as explicit answers to survey-based questionnaire eliciting big5 and other personality traits) of a user to determine user's personality a priori, and utilizes these ‘emotional descriptors’ for subsequent matching either with other users using weighted matching of dimensions, and/or being served relevant content based on the matched dimensions of the single user or for both first and second users. Further, in the existing prior art, determining the weighted matching of dimension and prioritizing is performed for individual users. However, in the proposed invention, determining the weighted matching of dimensions and prioritizing is performed with respect to another user. Further, the proposed method utilizes the distance calculation method to create the emotional profile and to prioritize based on the user preferences.

Prior art deals with one or more emotional descriptors (and not cognitive responses such as eye tracking measures). In other cases, prior art, computes preferences/profiles during run-time (not computed from predetermined profiles) and in many cases only pertain to non-physiological responses and mostly deals with a single user at a time. This invention is aimed at creating a method and system (1) to integrate across emotive, cognitive, and explicitly reported responses, (2) gathering and scoring individuals on “standard” content across demographic bases, country or geographical bases, and storing them as the user's emotional profile, and (3) to match the user with one or more users in appropriate applications. For example, in a dating application, individuals can be matched based on the compatibility levels; in education, students may be matched/better connect with teachers, or in a day care the nannies may be chosen based on the child's temperament, and so on. This type of matching using neuroscience responses (physiological, camera, eye tracking, voice) and self-report mechanisms using stored emotional profiles, and across users are not seen in current literature.

### BRIEF DESCRIPTION OF THE DRAWING(S)

FIGS. 1a and 1b, according to an embodiment of the present invention, is an overview of the system used to create emotional profile for the user to determine the overall personality of the user and matching the emotional profile with other user's emotional profile to determine close compatibility.

FIG. 2, according to an embodiment of the present invention, is a system overview used to implement the proposed invention.

FIG. 3, according to an embodiment of the present invention, is a flow-chart used to explain the process of generating emotional profile for the user and matching the emotional profile with other users considering different dimensions required in various applications.

FIG. 4, according to an embodiment of the present invention, is an emotional profile probe content structure with different levels of categorization.

FIG. 5, according to an embodiment of the present invention, the emotional profiles shared from various geographical proximities, is uploaded in a cloud-based connected environment.

### FIGURES—REFERENCE NUMERALS

90—Device used to collect sensor attributes and to receive Profileprobe content.

91—Various sensors used to collect attributes of a user.

92—Additional user information collected from various sources.

100—System used for implementing the proposed invention

101—Measuring physiological responses of a user based on the attributes collected for the user.

102—Profileprobe content created by measuring the neuro-signal-responses or the physiological responses of the user.

103—Emotional DNA profile created using the Profileprobe content

103a—Emotional DNA profile of User 1

103b—Emotional DNA profile of User 2

103c—Emotional DNA profile of User 3

103d—Emotional DNA profile of User 4

104—Explicitly specified information for the user

105—User's overall personality determined using the Emotional DNA profile and externally specified user information.

201—Profile probing sensor module

202—Emotional profile creation module

203—Emotional profile matching module

204—Emotional profile clustering module

205—Storage module

206—Controlling module

500—Cloud database

501a—Geographical proximity sharing the emotional profile EP-2

501b—Geographical proximity sharing the emotional profile EP-1

501c—Geographical proximity sharing the emotional profile EP-3

### DETAILED DESCRIPTION OF THE INVENTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical, and other changes may be made without departing from the scope of the embodiments. The following detailed description is therefore not to be taken in a limiting sense.

Throughout the document, the terms emotional DNA profile and emotional profile are used interchangeably.

In an embodiment, the term first user refers to a user owning a first device that is provided with a plurality of emotional measurement sensors for measuring the emotional parameters based on the type of stimuli received through the sensors. The term second user refers to the user other than the first user whose emotional profiles are matched with the first user's emotional profile using a variety of prioritized and weighted distance metrics for determining the overall personality of the user.

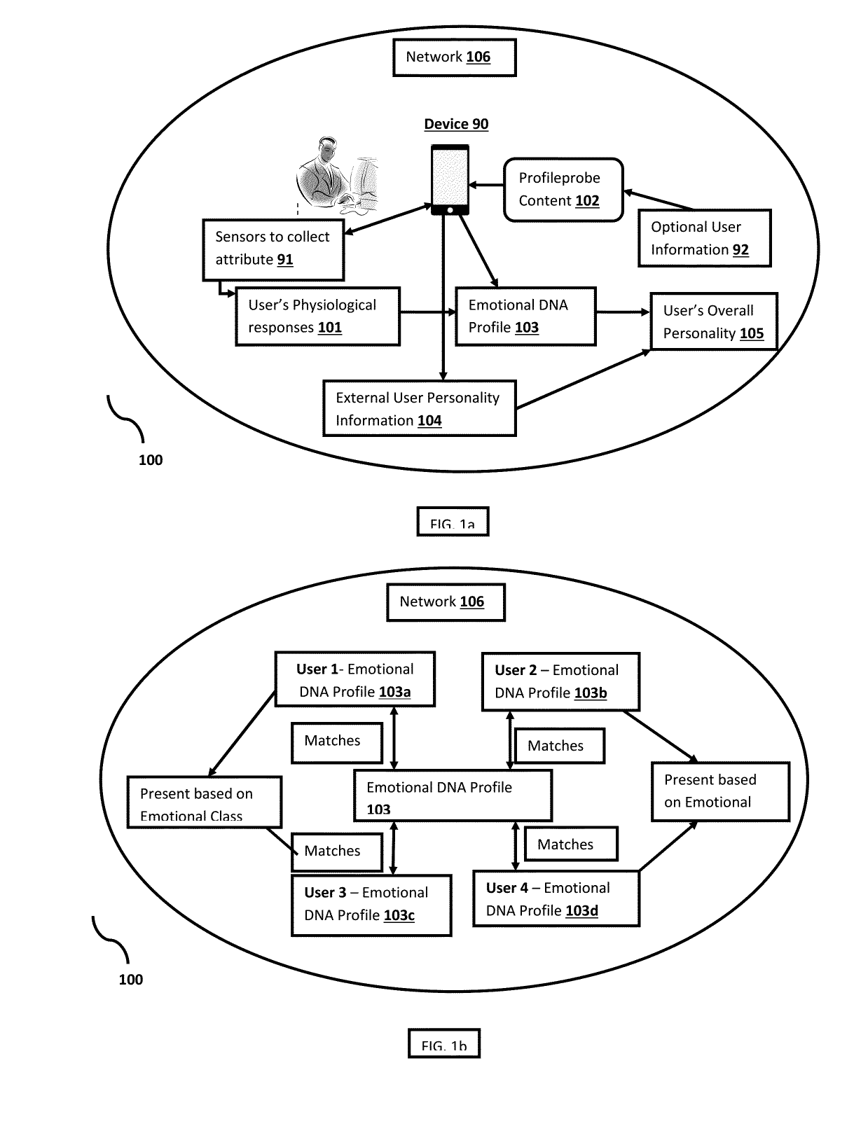

Referring to FIGS. 1a and 1b, depicts a working overview of the system 100 used to capture the overall personality of the user and to determine compatibility factor between users as accurately as possible.

In an embodiment, a ‘standard’ Profileprobe content (of stimuli) 102 is prepared and presented on a presentation device 90 to a user where the device 90 can be a desktop computer, a laptop, a smart phone, or any other medium capable of presenting audio/video stimuli. The user's physiological responses 101 (including, but not limited, to any subset of facial coding, voice-coding, eye tracking, pupil diameter, heart rate, skin conductance, and so on) are collected using a number of sensors 91. The sensors 91 are either built-in to the device such as a various types of cameras (for recording facial expressions, heart rate and so on), eye tracker, microphone) or/and optionally placed on appropriate places on the user for measurement (sensors for skin-conductance, heart rate, respiration and so on). Note that this is an example set of sensors but could be modified as the technology progresses to include more implicit monitoring of the user. As the Profileprobe content 102 is presented to the user, the responses are collected and converted into an emotional DNA profile 103. In an embodiment, the Profileprobe content 102 can measure all physiological responses including emotional responses as well cognitive responses (such as pupil diameter). In an embodiment, the emotional profile of the user determined by capturing the emotional responses as well as cognitive responses stimuli can be presented in the form of sequence of clips where each clip can be an image file, an audio file, or a video file, or in the form of real-life activities such as tasting food, enjoying food, promoting food, or other activities where emotive and/or cognitive responses of the participant may be measured. Further, a relevant subset of the responses can be measured for the Profileprobe content 102.

In an embodiment, the system 100 clusters the emotional profiles of a plurality of users and creates emotional personality segments/categorizations for the plurality of users. Further, the system 100 augments a Ten Item Personality Inventory (TIPI) and other behavioral indexes with the emotional personality segments/categorizations to provide a detailed behavioral characteristic of the user, which can be appropriately used in a variety of applications.

The emotional DNA profile 103, may or may not optionally, include explicitly reported personality measures (as in current literature/technology such as eHarmony, Match.com, Tinder). The user's sensitivity from a variety of emotion-eliciting content is received as responses 101 into the system 100 by using various neuroscience sensors 91 that capture user's unstated responses to the stimuli and collect attributes 101 associated with the content. For example: for a profile probe content 102, physiological responses 101 of the user such as facial coding responses (anger, fear, sadness, disgust, contempt, joy, surprise, positive, negative, confusion, frustration, anxiety), biometrics (skin conductance, heart rate, respiration), and voice expression responses, emotion from online activity (in face book, twitter and other sites, and forums) can be automatically measured. In an embodiment, the sensed inputs received from the sensors 91 can determine the interest level of the user in accordance with the type of genre. For example, by sensing the number of times a particular web site is visited by the user, the level of user's interest can be determined Further, based on these sensed inputs associated with the content, an emotional DNA profile 103 can be created specific to individual applications or for a generic application.

For example, the emotional DNA profile 103 and the corresponding Profileprobe content 102 created for a matching site may be different from the emotional DNA ‘profile’ 103 and the corresponding measuring Profileprobe content 102 created for interactive applications in a social-media. In another embodiment, standard generic probe content may be used for all applications and hence the emotional DNA profile 103 can be the same across all applications. In another embodiment, the Profileprobe content 102 may be the same but assigned with different weights to suit to different applications for creating various versions/flavors of the emotional DNA profile 103 for the user. Further, the method may continuously adapt the emotional DNA profile 103 as well as the Profileprobe content 102, from time to time, to capture specific dimensions needed for various applications. The method may adopt a mechanism to continuously refine the user's emotional DNA Profile 103 and the Profileprobe content 102 by learning most relevant content required for various applications. Further, based on the emotional DNA profiles 103 created for the user, augmented with external user information 104, the system 100 analyzes the overall personality of the user 105. Further, as depicted in FIG. 1b, the method utilizes the emotional DNA profile 103 to match across various users by appropriately combining the weights on the dimensions (either set by the system, or as an advanced option to be specified by the users). In an embodiment, the emotional DNA profile 103 dimensions are set by considering the physiological response dimensions, as well as the content dimensions (both time slices as well as content categories) and also by including the explicitly-reported personality dimensions. Here, only a subset of the dimensions may actually be used. The specific weighting of the dimensions may depend on the application. Further, based on the emotion DNA profile 103 matching, the method personalizes (that is, presents appropriate relevant) marketing content/products for the user by augmenting additional information on what interests each emotion class of the users. For example: if the emotional DNA profile 103 on a client/server contains attributes related to sports, entertainment, industry, and technology domains, and if the User 1 has a matching emotion DNA profile 103a related to sports, User 2 has a matching emotional DNA profile 103b related to entertainment, User 3 has a matching emotional DNA profile 103c related to industries, and User 4 has a matching emotional DNA profile 103d related to technology then appropriate content/product information is displayed to respective users based on the overall personality determined for the respective users.

In an embodiment, the emotional DNA profile 103 of the users can be shared with other users within the network 106 based on the user's preference. For example, if the user is busy or does not intend to share the emotional profile during lunch break then the method allows the user to configure the device to share the emotional DNA profile 103 when the user is not busy or post lunch break session.

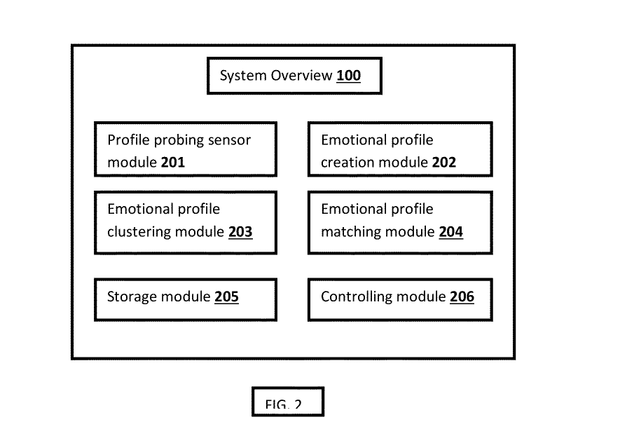

Referring to FIG. 2, depicts an overview of the system 100 used to implement the proposed method. In an embodiment, the system comprises of the following modules: a Profile probing sensor module 201, an Emotional profile creation module 202, an Emotional profile clustering module 203, an Emotional profile matching module 204, a Storage module 205, and a Controlling module 206. In an embodiment, the Profile probing sensor module 201 captures the emotional responses of the user from a variety of sensors including but not limited to one or more of: facial coding responses (anger, fear, sadness, disgust, contempt, joy, surprise, positive, negative, frustration and confusion), biometric responses such as skin conductance, heart rate, respiration, movement (accelerometer), and voice expression responses (for example: output from vendors like Cogitocorp, Beyondverbal or OpenEar), and/or emotion from online activity (in face book, twitter and other sites, and forums). In another embodiment of the invention, the emotional responses are allowed to include both emotive responses as listed above as well as cognitive responses such as pupil dilation, fixation time, first fixation and other measures from eye tracking. The emotional responses of the user can be captured from various types of genre that includes but not limited to movies, sports, art, hobbies, vacation preferences, personal preferences and activities to a user on a mobile device. Although cognitive measures may also be included in the measurement along with emotive responses, the profile is termed as an emotional DNA profile because emotive response matching is weighted higher than cognitive response matching in the system.

In an embodiment, the Emotional profile creation module 202 is configured to generate Emotional DNA profile 103 content that is tailored to specific deployment platform/application such as social media, or matching application or for specific cultures, or may be created as a generic content optimized for a variety of applications. This generic Emotional DNA profile 103 content may be refined over time to include (machine-learning based) knowledge on what content interests users over a set of applications over time. For example, for a matching application, the Emotional DNA profile 103 content can be customized based on the explicitly specified cultural background of the user. Alternately, the content could be generated as a generic content that may elicit interesting responses across a wide variety of users (irrespective of the user's background). In one embodiment, the Emotional DNA profile 103 will have content to determine a user's response ratings in the following categories (categories that capture various aspects of a lifestyle) including but not limited to:

Eating/Food Habits

Sleep and other Recreational Habits

Career

Entertainment: Movies, Sports, News, Sitcoms, Series

Daily Hobbies

Vacation Preferences

Family preferences

Overall Background

Online Activity

In contrast to all existing solutions where the information is gathered using explicit content, the user is not burdened with too many surveys to fill to collect all the information. In an embodiment, the user is allowed to just watch a generic (optionally tailored if needed based on culture, geography and other constraints in one embodiment of the invention) content and will have the system 100 analyze detailed information regarding the user's personality using physiological responses such as, which type of food the user likes/dislikes, which genre of movies they may like and what sections of a movie/trailer appealed to the user and so on.

In an embodiment, the Emotional profile clustering module 203 is configured to combine the profile of the user with profiles of other users to create a training dataset, and typical machine learning techniques (supervised or unsupervised clustering methods) are applied to identify user clusters. In an embodiment, the Emotional profile clustering module 203 is configured to cluster the emotional profiles of various users by adopting any of the existing machine learning techniques such as DBSCAN, Clarans, Kmeans, and the cluster attributes are explored to identify descriptive traits of the user that are common across each cluster.

Further, concise descriptions of those classes/clusters are tagged with the individual users for ease of use in catering targeted content to the user. In an embodiment, the Emotional profile clustering module 203 is configured to utilize the emotional DNA profile 103 across various participants in machine learning functions such as: clustering or classification: identifying specific emotion segment clusters or classes that capture a closed set of participants, Outlier detection: identifying which users are outliers in the database of emotion profiles. For example, to determine which users do not belong to any cluster. This can be used in screening participants in military, security clearance, flagging users for potential illegal activity and so on, creating various models to capture the semantics of the emotion clusters using supervised clustering (also known as classification) models such as decision trees, Bayesian models. In an embodiment, the emotion clusters or classes are trained/combined with behavioral data outcomes to refine and fine-tune the clusters, over various periods of time.

In an embodiment, the Emotional profile matching module 204 is configured to use the emotion profile (with and without additional explicitly collected personality dimensions of the user) in a variety of applications for matching with other users' profiles and determining compatibility. In one embodiment of the invention, the matching can be at the raw temporal traces of the various signals of the two users. In another embodiment, the raw signals may be aggregated to ratings of categorical sub-segments, or explicitly marked events. In one embodiment of the invention, the ratings of the explicitly marked segments/events as well as the raw temporal traces could be created as two facets of the same DNA and can be used in matching with others on either/or/both ‘facets’ of the DNA with appropriate weighting. In an embodiment, the Storage module 205 is configured to store the Profileprobe content 102 and the emotional DNA profile 103 on an electronic device/server.

In an embodiment, the Controlling module 206 can be configured to perform additional functionalities, such as generating/accessing the Profileprobe content 102, presenting it to a user and gathering physiological responses (including but not limited to one or more of facial coding responses (anger, joy, sadness, fear, contempt, disgust, surprise, positive, negative, frustration, confusion), eye tracking (for example: fixations, gaze and pupil diameter as indicator of cognitive responses), biometric responses (heart rate, skin conductance, respiration, motion), generating an emotional vectors for time slices from these responses and further creating an emotional DNA profile 103, transferring the emotional DNA profile 103 of the user to a server, determining the weight age of the attributes associated with specific applications for the Emotional DNA profile 103 content, determining the emotional-index matching score for the user to either a database of users or to a class of users, and the like.

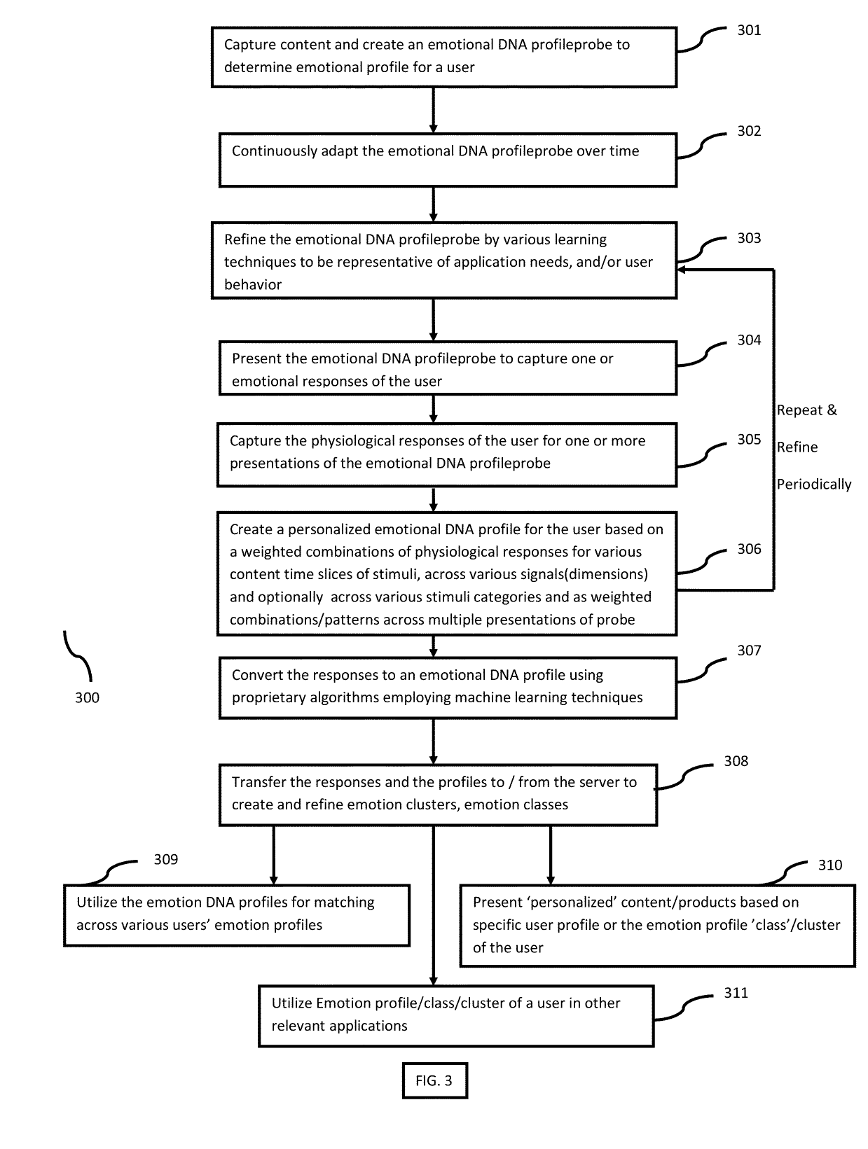

Referring to FIG. 3, depicts the process followed in determining the user's overall personality and matching the user's profile with other user's profile to determine compatibility level between users. Initially, at step 301 the method 300 creates Profileprobe content 102 to capture a user's sensitivity to a variety of standard emotion-eliciting content that can be used across a wide variety of users. In an embodiment, the Emotional profile creation module 202 creates the Profileprobe content 102 to capture the user's sensitivity to a variety of emotion-eliciting content. At step 302, the method 300 continuously adapts the emotional DNA Profileprobe 102 to capture specific dimensions required for various applications. In an embodiment, the Emotional profile creation module 202 can be configured to adapt the emotional DNA Profileprobe 102 to specific dimensions required for applications such as sports, interactive games, and so on. At step 303, the method 300 refines the emotional DNA Profileprobe 102 by adapting various learning techniques to get most relevant content for various applications. In an embodiment, the Controlling module 206 can be configured to refine the emotional DNA Profileprobe 102 by adapting various learning techniques to get most relevant content for various applications. At step 304, the method 300 presents the emotional DNA Profileprobe 102 (a set of video/audio/image clips) content that include but not limited to various types of genres in movies, art, hobbies, activities to a user on a mobile device to determine the emotional responses of the user based on the Profileprobe 102 content. At step 305, the method 300 starts capturing the emotion response from the Profileprobe 102 content that is created. In an embodiment, the Emotional profile creation module 202 can be configured to capture the physiological responses from the Profileprobe 102 content. At step 306, the method 300 creates a personalized emotional DNA profile 103 for the user based on a weighted combinations of physiological responses for various content time slices of stimuli, across various signals (dimensions) and optionally across various stimuli categories and as weighted combinations/patterns across. At step 307, the method 300 converts the emotion response determined from the Profileprobe 102 content and stores the emotion response in an emotional DNA profile 103 that is created for the user. In an embodiment, the Controlling module 206 can be configured to convert the emotional responses into an emotional DNA profile 103 structure by a using any of the existing algorithms/conversion techniques. In one embodiment, the responses of the user (for the time slices) are compared with the average responses of a set of users (in a database) and the deviations from the average (or normal) (in either direction, either above or below average) are marked as the distinctive directional traits (either above or below average) of the user's emotional DNA (or personality). At step 308, the method 300 transfers the emotional responses and the emotional DNA profile 103 to the server to create and refine one or more emotion classes. In an embodiment, the Controlling module 206 can be configured to transfer the emotional responses and the emotional DNA profile 103 to the server to create and refine one or more emotion classes. At step 309, various users' profiles 103 are matched with the emotional DNA profile 103 stored in the server to determine the compatibility level existing between users. In an embodiment, the Controlling module 206 can be configured to match the user's profile 103 with the emotional DNA profile 103 stored in the server to determine the compatibility level existing between users. At step 310, based on the level of matching determined for the user, appropriate content or product is displayed to the user. In an embodiment, the Controlling module 206 can be configured to display appropriate content or product to the user based on the level of matching determined for the user. For example, if the user's interest matches with the sports attributes then advertisements related to sport can be displayed to the user. At step 311, the emotional DNA profile 103 or the emotional classes of the user can be shared with other relevant applications for matching the user's profile with attributes relevant to the application. In an embodiment, the Controlling module 206 can be used to share the emotional DNA profile 103 or the emotional classes of the user with other applications.

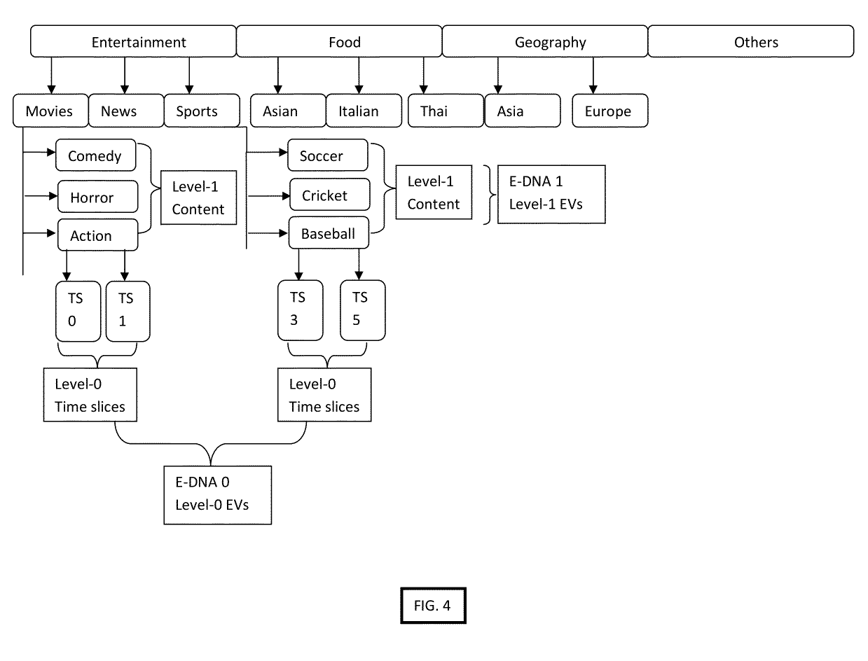

Referring to FIG. 4, depicts an emotional DNA profile 103 created for the user by considering different dimensions in various applications. As depicted in the figure, the emotional DNA profile 103 content will be a linear sequence of image/audio/video clips, where each clip presents content in one or more of the above categories. Each of these sub-clips are further divided into time slices (bins of time instances), for example, 5 s time slices (in another embodiment, the time slices could be of 1 s duration and in other cases, the duration of the time slices vary based on the signal). Example of an emotional DNA profile 103 content sequence (and the corresponding categories) is shown in the figure. The lowest level of content semantic hierarchy (for example: comedy, action, horror), is referred to as the level-1 categorization of the content. Each of the nodes at Level-1 is sub-divided into fixed-size or variable-size denoted as TS (1:N). In an embodiment, the physiological responses for the time instances are converted to standard ratings and aggregated to account for the width of each time slice. The time slices themselves may be defined based on temporal trace curves for the specific signal for each time slice. For each time slice TS (i), a vector of “k” physiological responses, called the Emotional Vector and denoted as EV (i, 1:K) are captured. In an embodiment, the emotional DNA profile content creates a matrix of numbers where columns correspond to time slices and rows correspond to physiological responses. The level-1 node may not correspond to an actual physical content clip but a temporal sub-segment of it. The temporal sub-segment may either represent a semantic sub-element of the content, or just a time slice/bin of the content (which may be used for matching purposes). In an embodiment, the emotional DNA profile content may use a combination of images, audio, and video to minimize the time that the user needs to watch to gather a high-level first version of their emotional-DNA. This emotional DNA can then be refined over time to get more refined details in various categories. In another embodiment of the invention, a directed acyclic graph (DAG) structure will represent the different parts of the emotional DNA profile content. For each Level-0 time slice in the emotional DNA profile content, there will be a set of user-level physiological responses that will be measured and stored as “Emotion vectors” and the entire set of EmotionVectors is referred to as the emotional DNA profile of the user (for the specific emotional DNA profile content). These responses may include but are not limited to: actual physiological dimensions that are measured including facial expression responses (such as joy, anger, disgust, contempt, sadness, fear, and surprise, and overall positive, negative). Other physiological measures, where relevant, optionally includes but not limited to zero or more of the following: vocal responses (such as tone, composure, mood, speaking rate, dynamic variation), eye tracking (gaze and fixation coordinates), skin conductance (to indicate arousal), heart rate (camera-based, or sensor-based). And/or any explicitly stated, or gathered online user behavior (for example, number of views). In an embodiment, the values for each of these could be normalized to a scale of 0 to 15 (4 bits) (or an appropriate range that is a power of 2, for ease of reference, we will stick with 0 to 15 for all dimensions although that need not be the case) for numerical dimensions (used in the temporal slices and other attributes) and as text for some specific categorical attributes, for example: food, (although textual categories could as well be discretized in numeric format for compactness of the structure). In one embodiment of the invention, for compact representation, the EmotionVector will consist of a series of binary bits where each 4 bit represents response information for one of the dimensions described above. An example can be the following where Joy dominates other emotions:
EmotionVector: 0110 (Anger), 1111 (Joy), 0000 (Sad)
For Level-0 time slices, the Emotion Vector EV can be represented as a matrix: EV (a,b)

Where A ranges from 1:N time slices and

B ranges from 1:K signals

This matrix is referred to as the Level-0 E-DNA (or the primary E-DNA unless otherwise mentioned).

In one embodiment of the invention, the emotional DNA profile can be generalized to higher levels, and E-DNA across various sub-units (such as temporal slices, or categorical sub-units) can be aggregated in meaningful ways to represent aggregate scores for the higher level nodes in the emotional DNA profile. This higher-level E-DNA will serve as the most concise description of an individual. The higher-level generalizations could be based on categorical hierarchy, or by just aggregating the time slices in meaningful ways to reduce the number (without explicitly being tied to semantic categories).

It is possible that the content duration may be divided differently across different signals: for example, for slow-moving signals such as GSR, the content may be divided into 5 s time slices; for HR, it may be divided into 2 s slices. In one embodiment of the invention, the content may be divided into time slice vectors TS(1:NK) where NK is the number of slices for signal ‘K’. The Emotion vectors will also be represented by

EV(a,b)

Where A ranges from 1:NK for the number of slices for each signal;

and B ranges from 1:K to denote the signal's response

Note that in this above case, EV will not be a matrix of number but a list of list of numbers.

In another embodiment of the invention, the emotion responses are normalized with the responses across the entire Emotional DNA profile 103 content by z-scoring and then scaling the range to the appropriate number of bits desired (e.g. 0 to 15, or 4 bits) for each dimension. This method works even in the absence of any training data model and scores against the Emotional DNA Profileprobe 102 itself. Since the Emotional DNA Profileprobe content 102 is used as a standard across a number of individuals, this method ensures consistent scoring for the dimensions.

In another embodiment of the invention, for some of the dimensions, the raw emotion responses (after doing a baseline deduction if employed) are used as is and the range is just scaled from a 0 to 1 to a 0 to 15 (or whatever desired maximum) as needed. This might be especially effective for facial coding responses where the responses are measured on a 0 to 1 scale and indicate the intensity of the response (from an expert's point of view).

In another embodiment of the invention, the ProfileProbe is divided into parts consisting of an orienting stimuli (responses for which may be discarded), baseline content, and then Probe-content which includes the various ‘content segments’ like sports, drama, and so on. The responses for the baseline content are used to transform the responses for the probe content to comparable levels across various users. For instance, the normal heart-rate ranges of various users may be at different levels; one user may have the heat range between 60-100 for most activities; another user may have the heat range between 140-200. Using the baseline content to measure the average (avg) and standard deviation (stddev) of the responses over the baseline content period, and utilizing such avg and stddev to transform each response value in the ‘probe’ content into a z-score will likely bring different users with varying physiology to similar levels. For example, a response value x(t) in probe content at time instant t can be transformed (or “normalized”) into z-scores (or T-scores) as:
Transformed_Z_x(t)=(x(t)−avg)/stddev;—This is zscore and will be typically in [−1,1] range but outliers could be much higher/lower and need to be scaled/binned accordingly.
Transformed_T_x(t)=Transformed_Z_x(t)*10+50—This is T-score and will be typically in [0,100] range but outliers could be higher/lower and need to be scaled/binned accordingly.

In one embodiment of the invention, the entire probe content itself is used as the baseline content (there is no separate baseline content).

In one embodiment of the invention, the orientation, the baseline, and the probe content may be interspersed in various time spans of the ProfileProbe content.

In one embodiment of the invention, for some or all of the dimensions, the normalized (z-scored) emotion responses (e) of a first user for each specific content segment of a ProfileProbe content is further “graded” by comparing it with corresponding responses for same or equivalent content of ProfileProbe of a database of second users using descriptive statistical techniques involving the average and standard deviation, or median and Inter-quartile range (IQR) of such responses as follows.
Grade(e)=ceiling((e−average)/(k*standarddeviation))

Where k is a number between 0.5 and 3

### OR

Grade(e)=ceiling((e−median)/(f*IQR))
Where f is a number between 0.5 and 3
   In this embodiment of the invention, the “grades” or “classes” directly constitue the response array (for the specific content segments) in the EmotionVectors of the emotional DNA profile of the user.

In one embodiment of the invention, for some or all of the dimensions, the normalized (z-scored) Emotion responses are fed into a machine-learning model that classifies the response output into as many classes or grades as needed (e.g., 0 to 15 if 15 classes are used in a 4-bit packet for a specific dimension) as EmotionVectors and emotional DNA profile for the user. This machine-learning model is computed by training using a set of emotion responses against an explicitly gathered target outcome set.

In another embodiment of the invention, for some or all of the dimensions, the raw responses may constitute the EmotionVectors of the E-DNA of the user.

In another embodiment of the invention, for some dimensions, the distance of the normalized response of the user from that of an expert (or to the average, or median of a panel of experts' (or chosen users') responses) is measured and that distance is inverted to represent a number in the 0 to 15 range, that is closer to an expert/average response, to get a high value (closer to 15), far away from expert/average response to get a low value (closer to 0). The EmotionVector value and the E-DNA could then be constructed using this set of computed values (based on distances to expert/average user response). It is possible that this method may mark the best in the above set of alternatives if target outcomes/training is not available.

In an embodiment of the invention, an analytical and data mining system may be built on a database of E-DNA profiles of various users. For example, for each dimension, the users that scored high or low on the corresponding EmotionVector may be identified and targeted with specific relevant material. Alternately, the database could be used to match with other users that have “compatible” E-DNA wherein the compatibility is user-defined or system-defined using a system of distances and weights.

In one embodiment of this invention, the database of E-DNA profiles may be appropriately combined for analysis and mining with other available information of the users such as geographic location (either explicitly entered and/or implicitly tracked by location-tracking embedded in the user's device), personality dimensions (TIPI or other), user preferences, past history and other available information. For example, the database analyzed either by geographic location or by emotionprofile dimension, or a combination thereof, or by other standard analytical approaches. The results of such analyses may be plotted into appropriate dashboards called emotion-profile maps. One approach for such maps is to use standard geographic boundaries to analyze the emotion-profiles. For example, within each geographic region (where region is an appropriate aggregation of the locations as utilized in standard maps and GIS terminology), the E-DNA profiles may be examined and the top-few dimensions that have high-scores (above a specified threshold) or alternately low-scores (below a specified threshold) for a majority of users (say at least a substantial portion of the E-DNA users in that region) may be determined to color-code the geographic region in “emotion profile dominance-maps”. Alternately, in another embodiment of the invention, for each dimension, the high scoring (or alternately low scoring) profiles that are above a threshold value for that dimension may be plotted based on their geographic location. Standard clustering techniques from machine learning may be employed to determine tight-clusters of high- (or low-) score users for the specific dimension. These may be referred to as high-score or low-score geographic-cluster maps for that specific dimension and may identify concentrations of geography for that emotion-dimension where a lot of users that score high or low. In one embodiment of the invention, the (high-score or low-score) cluster maps of multiple emotion dimensions may be merged to identify emotion cluster-impact maps.

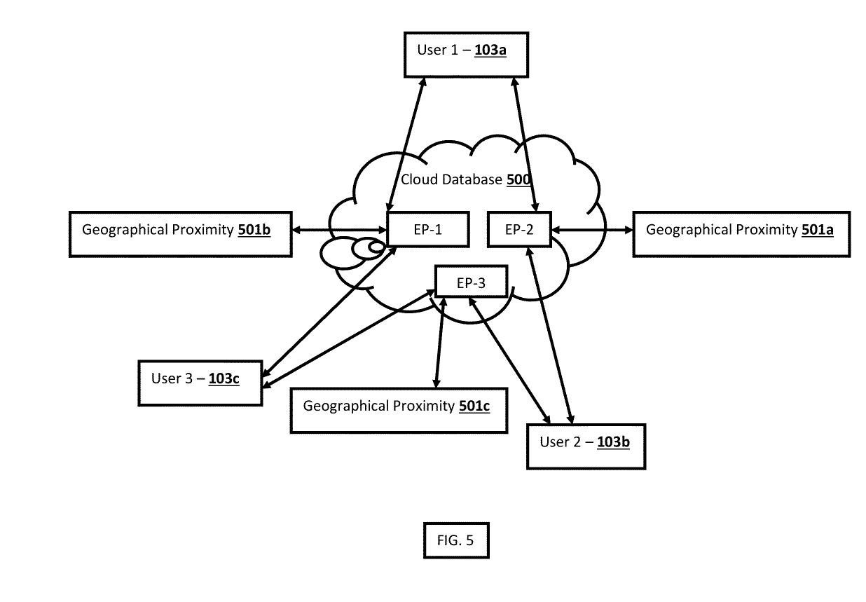

Referring to FIG. 5, depicts the emotional profiles shared from various geographical proximities uploaded in a cloud-based connected environment 500. In an embodiment, the cloud-database 500 stores the emotional profiles EP-1, EP-2, and EP-3 that are shared from various geographical proximities 501a, 501b, and 501c respectively. The method notifies the existence of emotional connections in the geographical proximity while concealing the true identities of the connections and optionally reveals/allows the user to browse and choose the various matching and unmatching personality dimensions of the connections before revealing and introducing the connections. For example, User 1 103a can optionally access information about the emotional profile EP-1 and EP-2 connected to the geographical proximities 501a and 501b and determine the matching personality dimensions considering the two emotion profiles EP-1 and EP-2 shared through the connected environment.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

1. A system for creating and matching the emotional DNA profile of a user considering at least one type of content, wherein the system comprising of:
a concise set of stimuli, known as ProfileProbe, that includes a plurality of image, video, and auditory content to assess normalized interest and engagement levels of audiences in various aspects relevant to an application;
a plurality of emotional measurement sensors in a first device operable to measure a plurality of emotive and/or cognitive parameters for a first user of the first device when exposed to at least one type of stimuli from the ProfileProbe content;
a computer system that converts the raw emotional responses of the first user to various (segments or) dimensions in the said ProfileProbe content into a normalized, graded set of EmotionalVectors that together constitute the emotional profile of the first user;
a computer system operable to match the emotional profile of said first user with a database of the emotional profile of at least one second user and returning a ranking of said at least one second user based on the multi-dimensional proximity of the emotional profile associated with at least one second user, which is determined using at least one prioritized and weighted distance metrics;
a means to provide an option for the first user to share the emotional profile with said at least one second user within the system based on the first user's preference;
a computer system that clusters or classifies the emotional profiles of a plurality of users and creates emotional personality segment classes/clusters for said plurality of users;
a computer system that can augment a Ten Item Personality Inventory (TIPI) and other behavioral indexes with the emotional personality segments/categorizations to provide a detailed behavioral characteristics for said plurality of users that can be used appropriately in a variety of applications.
a computer system that can exploit the emotional personality indexes and emotional class or cluster labels of said user to serve targeted content as needed or match with other users;
a computer associating system to identify and notify the existence of emotional connections in the geographical proximity while concealing the true identities of the connections;
a means to optionally reveal/allow the first user to browse and choose the various matching and unmatching personality dimensions of the connections before revealing and actually introducing the connections; and
a computer system wherein the database of emotional DNA profiles can be appropriately combined for analysis and mining with other available information of the users such as geographic location (either explicitly entered and/or implicitly tracked by location-tracking embedded in the user's device), personality dimensions, user preferences, past history and other available information.

2. The system as claimed in claim 1, wherein the type of content considered for creating the emotional DNA profile can be captured from at least one type of genre that interests wide range of users using a standard scoring mechanism and said at least one type of genre can be one of: a movie, a sport, an art, a vacation preference, a personal preference, career, food habits, daily hobbies, or the like.

3. The system as claimed in claim 1, wherein the type of stimuli used to measure said plurality of emotional parameters to determine the emotional profile of said user comprises of capturing emotional responses as well as cognitive responses presented in the form of sequence of clips, where each clip can be an image, an audio file, or a video file, or from real-life activities such as tasting food, enjoying food, promoting food, or other activities from which emotive and/or cognitive responses of the participant may be measured.

4. The system as claimed in claim 1, wherein the plurality of emotional parameters that are measured include but not limited to one or more of electrodermal activity (skin conductance, resistance etc), heart rate activity (heart rate, heart rate variability etc), respiration, facial coding responses (neutral, anger, fear, sadness, joy,surprise, disgust, contempt, positivevalence, negativevalence, confusion, frustration, anxiety etc), eyetracking responses (pupil dilation, timetofirstfixation, other attention measures, etc), movement (accelerometer responses from various parts of the body or device), geolocation (built-in gps responses), blood pressure and blood oxygen levels, EEG, EMG, fMRI, voice emotion responses (speechrate, variation, emotiontype etc.) and explicit self-report-based personality, preference responses.

5. The system as claimed in claim 1, wherein the first device that collects the plurality of emotional parameters involves one or more sensors and/or accompanying software capable of measuring these emotional parameters wherein the said sensors may be embedded either internally in the device or externally attached to the device to augment the capabilities of the said device to measure the said emotional parameters

6. The system as claimed in claim 1, wherein the raw emotional responses of the first user to various (segments or) dimensions in the said ProfileProbe content are normalized, and graded into a responsearray of EmotionalVectors that together constitute the emotional profile of the first user;

7. The system as claimed in claim 6, wherein the emotional profile of a first user, along with additional ‘outcome’ data including behavioral information (such as usage, activity, weblogs, patterns) and other relevant information of a first user, is transferred and managed in the cloud by one or more computing servers and one or more storage servers, cumulatively referred to as the cloud-server.

8. The system as claimed in claim 7, wherein the cloud-server creates, updates and manages a database of emotion profiles of various users and applies machine-learning techniques on the emotion profile database with and without the outcome behavioral data (as target variables).

9. The method as claimed in claim 8, wherein the machine-learning methods are unsupervised clustering techniques used for exploring and utilizing common descriptive traits (of user profiles in each cluster) for use in specific applications both on the server and the client devices wherein such cluster information is propagated. The descriptive traits may be named (or labeled) appropriately for easy identification and for matching by the named descriptive traits verbally.

10. The method as claimed in claim 8, wherein the machine-learning techniques are supervised classification or regression techniques utilizing emotion profile database and behavior data for creating emotion-profile machine-learning models and utilizing such models to either assign one or more ‘emotion class labels’ to a user or to predict outcome behavior variables for the emotion profile of the said user and utilizing such class labels or outcome variables to drive the experience of the user in a said application or to match with other relevant users. The emotion classes may be named or labeled for ease of identification and matching with other users.

11. The system as claimed in claim 1, wherein the emotional DNA profile created for said user can be represented in the form of a matrix, a directed acyclic graph, an emotional vector, an aggregate scoring level, a range of classes, or the like as required by the application.

12. The system as claimed in claim 11, wherein the emotional DNA profile dimensions are set by considering the physiological response dimensions, the content dimensions, and the explicitly-reported ‘personality’ dimensions as well as additional lifestyle traits such as sleeping habits, eating traits, and other explicitly-reported preferences.

13. The system as claimed in claim 1, wherein the system is configured to generate emotionprofile dominance maps and emotion cluster impact maps by performing analysis and mining on the database of the emotional DNA profiles.

14. A method for creating and matching the emotional DNA profile of a user considering at least one type of content, wherein said method comprises of:
capturing a plurality of emotional parameters for a user of the first device when exposed to various types of stimuli using a plurality of emotional measurement sensors in a device;
converting the raw emotional responses of a first user to various (segments or) dimensions in the said ProfileProbe content into a normalized, graded set of Emotional Vectors that together constitute the emotional profile of said first user;
matching the emotional profile of the first user with a database of the emotional profiles of at least one second user and returning a ranking of said at least one second user based on the multi-dimensional proximity of the emotional profiles of the various second users to the emotional profile of the first user using at least one prioritized and weighted ‘distance’ metrics;
clustering or classifying the emotional profiles of various users and creating emotional personality segment classes/clusters for users;
augmenting TIPI and behavioral indexes with the emotional personality segment classes/clusters to provide a detailed behavioral characteristics of said user for appropriate use in a variety of applications;
utilizing the emotional personality indexes and emotional class or cluster labels of said user to serve targeted content as needed or match with other users;
identifying and notifying the existence of emotional connections in the geographical proximity while concealing the true identities of the connections; and
optionally revealing/allowing the first user to browse and choose at least one matching and unmatching personality dimensions of the connections before revealing and introducing the connections,
applying a method or set of methods on the database of emotional DNA profiles that are appropriately combined for analysis and mining with other available information of the users such as geographic location (either explicitly entered and/or implicitly tracked by location-tracking embedded in the user's device), personality dimensions, user preferences, past history and other available information.

15. The method as claimed in claim 14, wherein the type of content considered for creating the emotional DNA profile can be captured from at least one type of genre that interests said user and said at least one type of genre can be one of: a movie, a sport, an art, a vacation preference, a personal preference, career, food habits, daily habits, sleeping times, durations, or the like as required by the application.

16. The method as claimed in claim 14, wherein the type of stimuli used to measure said plurality of emotional parameters to determine the emotional profile comprises of capturing emotional responses as well as cognitive responses presented in the form of a sequence of clips where each clip can be an image file, an audio file or a video file or from real-life activities such as tasting food, enjoying food, promoting food, or other activities where emotive and/or cognitive responses of the participant may be measured.

17. The method as claimed in claim 14, wherein the raw emotional responses of the first user to various (segments or) dimensions in the said ProfileProbe content are normalized, and graded into a responsearray of EmotionalVectors that together constitute the emotional profile of the first user;

18. The method as claimed in claim 17, wherein the emotional profile of a first user, along with additional outcome data including behavioral information (such as usage, activity, weblogs, patterns) and other relevant information of a first user, is transferred and managed in the cloud by one or more computing and storage servers, cumulatively referred to as the cloud-server.

19. The method as claimed in claim 18, wherein the cloud server creates and manages a database of emotion profiles of various users and applies machine-learning techniques on the database of emotion profiles with and without the outcome data as target variables.

20. The method as claimed in claim 19, wherein the machine-learning methods are unsupervised clustering techniques used for exploring and utilizing common traits (of user profiles in each cluster) in specific applications both on the server and the client devices wherein such cluster information is propagated.

21. The method as claimed in claim 19, wherein the machine-learning techniques are supervised classification or regression techniques utilizing emotion profile database and behavior data for creating emotion-profile machine-learning models and utilizing such models to either assign one or more emotion class labels to a user or to predict outcome behavior variables for the emotion profile of the said user and utilizing such class labels or outcome variables to drive the experience of the user in a said application or to match with other relevant users.

22. The method as claimed in claim 14, wherein the emotional DNA profile created for said user can be represented in the form of a matrix, a directed acyclic graph, an emotional vector, an aggregate scoring level, a range of classes, or the like.

23. The method as claimed in claim 24, wherein the emotional DNA profile dimensions are set by considering the physiological response dimensions, the content dimensions, and the explicitly-reported personality dimensions.

24. The method as claimed in claim 14, wherein the method generates emotionprofile dominance maps and emotion cluster impact maps by performing analysis and mining on the database of emotional DNA profiles.
